(19) United States
(12) Patent Application Publication
(10) Pub. No.: US 2004/0254572 A1
(43) Pub. Date: Dec. 16, 2004

(54) Self anchoring radio frequency ablation array

(76) Inventors: Jon T. McIntyre, Newton, MA (US); Barry N. Gellman, N. Easton, MA (US); Jozef Slanda, Milford, MA (US)

Correspondence Address: FAY KAPLUN & MARCIN, LLP, 15O BROADWAY, SUITE 702, NEW YORK, NY, 10038, US

(21) Appl. No.: 10/832,556

(22) Filed: Apr. 26, 2004

Related U.S. Application Data
(60) Provisional application No. 60/465,625, filed on Apr. 25, 2003.
(60) Provisional application No. 60/523,225, filed on Nov. 18, 2003.

(51) Int. Cl. A61B 18/14

(52) U.S. Cl. 606/41

(57) ABSTRACT

A system for tissue ablation includes a handle, a tissue anchoring portion operatively connected to the handle, the tissue anchoring portion forming an electrode of a first polarity and a central post or a plurality of arms deployable in proximity to the anchoring portion, at least the post or one of the arms comprising a second electrode of a second polarity. A method of ablating target tissue, comprises the steps of positioning a distal end of an elongated shaft so that it abuts the target tissue and anchoring the distal end in the target tissue by actuating an anchoring portion of the elongated shaft (e.g., a coil-like anchor) in combination with deploying the center post in the tissue or the steps of deploying an array of tines from the distal end of the shaft to contact the target tissue and applying an electric potential between a first electrode of the anchoring portion and a second electrode formed by the center post or the array of tines.

### CLAIM PRIORITY

Priority is claimed to U.S. Provisional Patent Application Ser. No. 60/465,625 filed Apr. 25, 2003 “RF Myoma Ablation”, and U.S. Provisional Patent Application Ser. No. 60/523,225 filed Nov. 18, 2003 “RF Ablation and Fixation Device”. The entire disclosure of these prior applications is considered as being part of the disclosure of the accompanying application and is hereby incorporated by reference herein.

### BACKGROUND

Uterine fibroids are among the most common tumors found in women, with symptoms which include severe pain and excessive menstrual bleeding. Current therapeutic procedures for treatment of these fibroids include removal of the uterus, or treatment by drugs (e.g., GnRH agonists), resection, interstitial RF ablation and open or laparoscopic surgery.

Once the presence of a fibroid has been ascertained, local ablation of the diseased tissue may be carried out by inserting a therapeutic device into the tissue and carrying out therapeutic activity designed to destroy the diseased cells. For example, electromagnetic energy may be applied to the affected area by placing one or more electrodes into the affected tissue and by discharging electric current therefrom to ablate the tissue. Alternatively, solids or fluids with appropriate properties may be injected to the vicinity of the affected tissue to chemically necrose and shrink selected portions of the tissue. RF ablation methods are especially well suited to treat tumors, because the tumor cells are not cut, and the incidence of seeding is greatly reduced. In addition, healthy tissue surrounding the tumor can be spared damage, since the RF energy dissipates rapidly before causing necrosis of the healthy cells.

Many tumors and fibroid tissues comprise very hard masses that are not securely anchored in place within the body, but instead are loosely held in place by ligaments and other structures. Accordingly, it may be difficult for a surgeon to insert an electrode into the target tissue as the tissue may move when the surgeon attempts to puncture it with the electrode. Grasping devices and anchors may be used to immobilize the tissue while an electrode is inserted thereinto, but these procedures add more complexity to the operation and may require additional incisions. The surgeon may also require assistance from additional personnel to carry out such procedures.

### SUMMARY OF THE INVENTION

The present invention is directed to a system for tissue ablation comprising a handle, a tissue anchoring portion operatively connected to the handle, the tissue anchoring portion forming an electrode of a first polarity and a plurality of arms deployable in proximity to the anchoring portion, at least one of the arms comprising a second electrode of a second polarity.

The present invention is further directed to a method of ablating target tissue comprising the steps of positioning a distal end of an elongated shaft so that it abuts the target tissue and anchoring the distal end in the target tissue by actuating an anchoring portion of the elongated shaft in combination with the steps of deploying an array of tines from the distal end of the shaft to contact the target tissue and applying an electric potential between a first electrode of the anchoring portion and a second electrode formed by the array of tines.

In another aspect, the present invention is directed to a thermal ablation apparatus comprising a first (e.g., positive) electrode assembly adapted for insertion in a body lumen or cavity, an elongated shaft of the first electrode assembly, a coil-like electrode mounted distally on the first electrode assembly, a second (e.g., negative) electrode assembly having an elongated shaft adapted for insertion in a working channel of the first electrode assembly, and an electrode mounted distally on the second electrode assembly, the electrode extending through the coil-like electrode. The exemplary use of the ablation apparatuses described herein includes treatment of fibroid tumors, but other applications, for example, other tumors, which have characteristics that lend themselves to the device configurations of the invention are contemplated. Also, while the exemplary treatment described here is the ablation of a target tissue mass in order to kill or necrose the tissue and shrink the mass, other degrees of treatment that may or may not result in ablation dependent, for example, on the amount of power, temperature reached, or time of treatment, are contemplated.

### BRIEF DESCRIPTION OF THE DRAWINGS

FIG. 1 shows a schematic diagram of a tissue ablation device according to an embodiment of the present invention, as positioned within a patient;

FIG. 2 shows a schematic diagram of a distal end of the tissue ablation device shown in FIG. 1;

FIG. 3 is a perspective view showing a positive electrode assembly according to a different embodiment of the invention;

FIG. 4 is a perspective view showing a negative electrode assembly according to a different embodiment of the invention;

FIG. 5 is a side view of the positive electrode assembly shown in FIG. 3;

FIG. 6 is a side view of the negative electrode assembly shown in FIG. 4;

FIG. 7 is an enlarged side view of a distal tip of the positive electrode shown in FIG. 3;

FIG. 8 is an enlarged side view of the distal tip shown in FIG. 7 partially entering a tissue;

FIG. 9 is a schematic drawing of a monopolar embodiment of a device according to another embodiment of the invention;

FIG. 10 is a schematic drawing of a bipolar embodiment of the device according to the invention;

FIG. 11 is a perspective schematic view showing an RF ablation device and endoscope assembly according to an embodiment of the invention;

FIG. 12 is a side view showing a positive electrode of the device shown in FIG. 11;

FIG. 13 is a side view showing a negative electrode of the device shown in FIG. 11;

FIG. 14 is a pictorial representation of a distal end of a tissue ablation device according to another embodiment of the invention;

FIG. 15 is a pictorial representation of different distal ends of positive electrodes and of a negative electrode according to embodiments of the present invention;

FIG. 16 is a pictorial representation of an RF thermal ablation device including a control console according to an embodiment of the invention;

FIG. 17 is a pictorial representation showing the distal ends of an insulated positive electrode and of an insulated negative electrode according to embodiments of the invention;

FIG. 18 is a pictorial representation of a front loaded positive electrode assembly according to the invention;

FIG. 19 is a first pictorial representation of a laboratory test conducted with an RF ablation electrode according to an embodiment of the invention; and

FIG. 20 is a second pictorial representation of a laboratory test conducted with an RF ablation electrode according to the invention.

### DETAILED DESCRIPTION

The present invention may be further understood with reference to the following description and the appended drawings, wherein like elements are referred to with the same reference numerals. The present invention is related to medical devices used to treat diseased tissue less invasively. In particular, the present invention relates to devices for ablating diseased or abnormal tissue using electric energy provided through a needle-like device which is inserted into target tissue.

Embodiments of the present invention may be used to treat diseased tissue via procedures less invasive than traditional surgical procedures. For example, the exemplary system may be used to necrose and shrink tumors and fibroid tissues on the walls of body lumens or cavities, such as uterine fibroids and similar growths. The electrodes used to deliver electrical current to the target tissue as well as the devices used to grasp and hold in place the target tissue are both deployable from the same medical instrument. Only one incision or puncture is thus necessary to perform the medical procedure and this procedure can be carried out with a reduced number of operators. This simplification and reduction in required personnel provides a significant improvement over conventional techniques, where the use of multiple medical tools results in a more complex and resource intensive procedure.

Conventional systems for ablating diseased tissue with needle-based devices include, for example, the LeVeen Needle Electrode™ from the Oncology Division of Boston Scientific Corp. and the Starburst™ product line available from RITA Medical Systems, Inc. When using these devices, the surgeon punctures the target tumor with the device's needle and then deploys one or more radio frequency (RF) tines into the tissue mass. An electric voltage is then applied to the tines to destroy the target tissue.

It requires great skill to use these devices because the tissue mass of the tumor or fibroid can move as the surgeon attempts to puncture it with the needle. The tissue mass is loosely held in place by ligaments or connective tissues, so that it can move relative to the surrounding tissues. Multiple attempts may thus be required before the needle is positioned correctly, prolonging the procedure and consuming valuable surgeon time. Alternatively, a grasping device such as a tumor screw may be used to immobilize and apply traction to the diseased tissue while the needle is inserted. This approach simplifies insertion of the needle into the tissue, but increases the complexity of the overall procedure—especially if multiple entry points through the skin are used to position the grasping device and the needle. Moreover, these procedures require the surgeon to manipulate multiple devices simultaneously, and may require the assistance of other personnel to complete the operation.

Many conventional RF tissue ablation devices are monopolar, meaning that electrodes of only one polarity are inserted into the target tissue during the procedure. To complete the circuit and cause current to flow through the tissue, one or more secondary grounding pads are provided in the vicinity of the target tissue on an outer surface of the skin to provide a second electrode. Such monopolar RF ablation devices can at times cause burns to the patient (e.g., at the grounding pads) which may further complicate recovery. Monopolar delivery systems may also require increased energy delivery times to achieve a desired level of tissue necrosis. Although not optimally efficient, monopolar RF ablation devices are used extensively because they operate with only a single electrode inserted per incision, simplifying the procedure.

The tissue ablation system according to one aspect of the present invention combines a radio frequency (RF) array of tines with an anchoring coil to form a device for the therapeutic treatment of target tissue such as fibroids or tumors. In one exemplary embodiment, the anchoring coil used to stabilize the target tissue and to facilitate insertion of the needle also serves as a one of the poles of a bipolar RF system, with the tines forming the other pole. This design offers the advantages of stabilization of the target tissue during insertion of the needle and deployment of the tines, as well as the increased efficiency and other benefits of delivering the RF energy through a bipolar electrode arrangement. Additional grounding pads are not required when using the system according to the invention and the associated burns are eliminated. In addition, the electrical energy delivery time is considerably shortened as compared to procedures using monopolar systems.

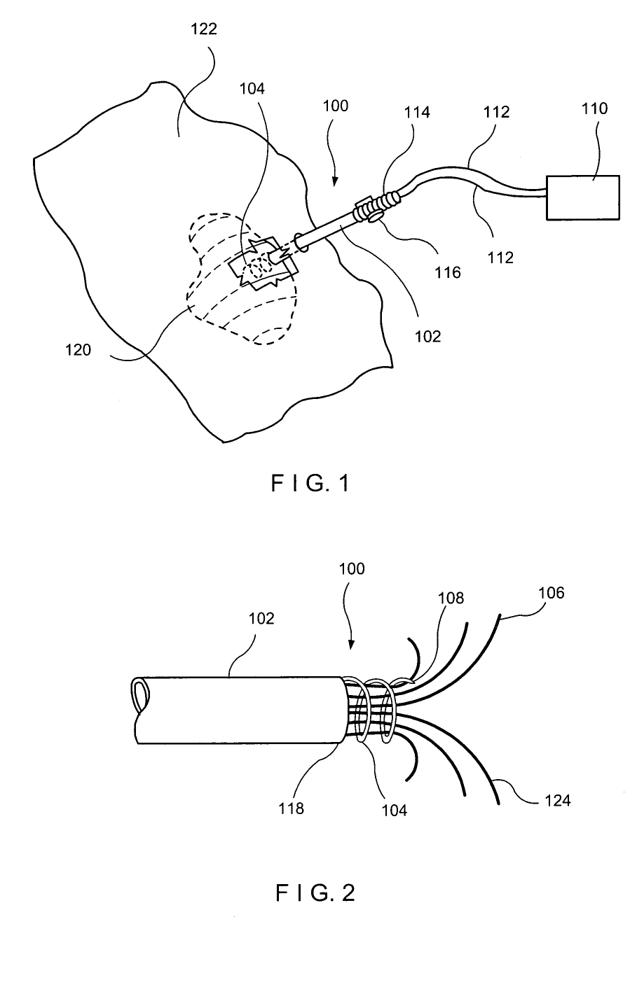

FIG. 1 shows an exemplary embodiment of a self anchoring RF array according to the present invention. In the drawing, a self anchoring RF ablation device 100 is shown inserted into target tissue 120 (typically a fibroid mass or tumor) through the patient's skin 122. Alternatively, the ablation device 100 may be inserted through a body lumen or cavity and may be placed in contact with the target tissue 120 within the lumen or in the lumenal or cavity wall. It will be apparent to those of skill in the art that the RF ablation device 100 may include different types of handle portions used to manipulate the device 100, and multiple controls to actuate the various functions of the device 100. A power supply 110 may be connected to the RF ablation device 100 through wires 112, so that a battery, AC adapter, or other source of power for the bipolar electrode array can be located remotely from the operating area.

As shown more clearly in FIG. 2, the exemplary embodiment of the RF ablation device 100 according to this aspect of this invention may include a shaft 102 having a distal end 118 which may be inserted into the patient through an incision or a perforation of the patient's skin 122, or through a naturally occurring orifice into a body lumen or cavity. Depending on the type of procedure being carried out, the shaft 102 is then pushed through tissues or through the body lumen until a distal end 118 thereof abuts the target tissue 120. A grasping device such as a coil 104 may then be used to anchor the RF ablation device 100 to the target tissue 120. In the exemplary embodiment, the coil 104 is fixed to the shaft 102. However, in a different embodiment, a grasping device similar to the coil 104 may be retracted into the shaft 102 (e.g., during insertion and removal of the device from the body) and may be extended from the shaft 102 when in the proximity of target tissue 120. In the exemplary embodiment, the coil 104 has a pointed end 108 which pierces the target tissue 120 as the coil is turned by rotating the shaft 102 along a longitudinal axis thereof.

A plurality of arms defining an electrode of the device may be used to form the bipolar system according to the invention. For example, an array of tines 106 is deployable from the shaft 102 once the device has been securely anchored to the target tissue 120. In the exemplary embodiment, the tines 106 are designed to extend from the hollow center of the shaft 102 and to be deployed through the center of the coil 108 along a longitudinal axis thereof. The tines 106 may also be designed to curve around the coil 108 in an umbrella-like fashion, partially surrounding the distal end of the coil 108. The tines 106 preferably comprise pointed ends 124, designed to easily penetrate into the target tissue 120. Alternatively, the system may be actuated with current flowing through the tunes in order to aid in penetration of the tines as they are deployed. After the tissue ablation procedure has been completed, the tines 106 may be withdrawn into the shaft 102 to facilitate removal of the RF ablation device 100 from the body. As would be understood by those skilled in the art, a sliding control knob 116 or other similar control device may be used to mechanically move the tines 106 out of and back into the hollow passage of the shaft 102.

Each of the tines 106 may be configured such that they can be actuated individually, in combinations of less than all of the tines, or all together to form a first pole of the RF ablation device 100, with the coil 108 forming a second pole of different polarity. When actuated, electric energy flows between these first poles and the second pole for delivery to the target tissue 120 located therebetween. The boundaries of a lesion formed by the electric energy within the target tissue 120 is controlled by positioning the deployed array of tines 106 around the coil 108 with the shape and relative position of the coil 108 and the tines 106 being selected to achieve a desired lesion location, shape and size, etc. in the target tissue. The RF ablation device 100 is suitable for forming a large area of necrotic tissue because the flow of energy is contained to the area of tissue between the tines 106 and the coil 108 and does not need to pass through intervening tissue to an external grounding pad.

In an exemplary embodiment, the shaft 102 of the RF ablation device 100 is formed of a biocompatible metal, such as stainless steel. The coil 108 may be made of the same material, or of another biocompatible metal which is a good conductor of electric energy. The tines 106 are also preferably made of a bio-compatible metal which is a good electric conductor. In addition, the material of which the tines 106 are made is also preferably flexible to enable the tines 106 to be deployed from and retracted into the shaft 102. It will be apparent to those skilled in the art that different materials and configurations of the coil 108 and of the tines array 106 may be used, depending on the shape and strength of the electric field that is required between the two electrodes. In this manner, the effective region of the bipolar RF ablation device 100 may be shaped and modified by selecting appropriate shapes of the two poles.

After a decision to treat a tumor or fibroid has been made, the RF ablation device 100 is configured for insertion into the patient's skin with the tines 106 retracted into the shaft 102 and the shaft 102 is inserted to the target tissue (e.g., through the patient's skin via a small trocar incision) until the distal end 118 thereof abuts the target tissue 120. The coil 108 is then inserted into and anchored to the target tissue 120, for example by applying a twisting, screw-like motion to the shaft 102. When the coil 108 is sufficiently secure in the target tissue 120, the tines 106 are deployed from the shaft 102 to a desired configuration relative to the coil 108 and the target tissue 120. The flow of electric energy between the coil 108 and the tines 106 is then begun. Once a lesion of sufficient size has been formed in the target tissue 120, the electric current is stopped and the tines 106 are withdrawn into the shaft 102. The coil 108 is then unscrewed from the target tissue 120 and the RF ablation device 100 is removed from the patient's body.

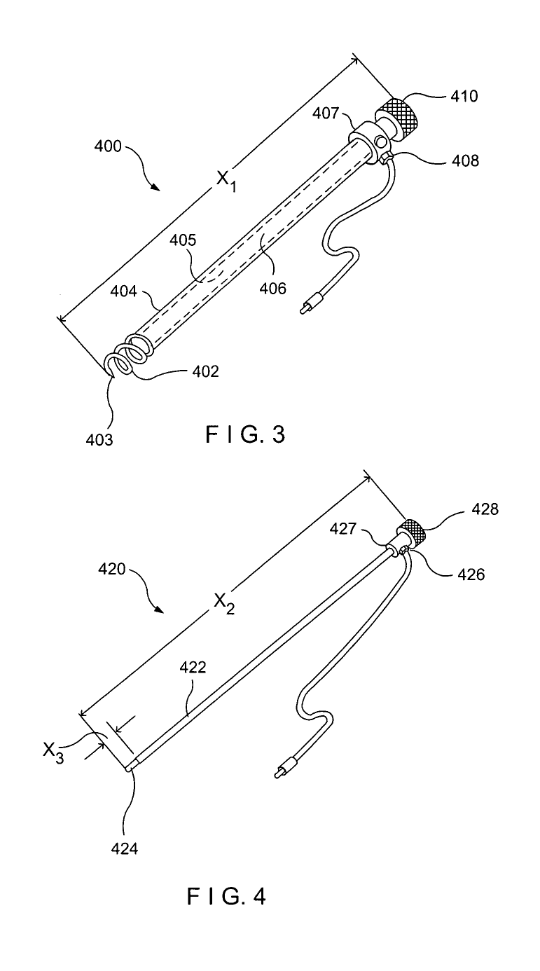

In a different exemplary embodiment according to another aspect of the present invention, the RF ablation electrodes may be formed into a complete medical tool which is insertable alone or into a catheter under independent external and/or internal imaging guidance, or though a scope (allowing for direct visualization), to reach the diseased tissue. The medical tool may include hand operated controls and electrical connections for separate positive and negative electrodes. For example, FIG. 3 depicts one embodiment of a positive electrode assembly 400 in accordance with the invention. The electrode assembly 400 comprises a coil electrode 402 coupled to a drive shaft 404. The electrode 402 can be coupled to the drive shaft 404 by welding, soldering, or other conventional methods. In a particular embodiment, the drive shaft 404 has an axial lumen 405 extending longitudinally along the drive shaft 404. In the embodiment shown, the drive shaft 404 is a stainless steel tube covered with insulation 406, which may comprise a polyamide heat shrink tube. Various materials and configurations for the drive shaft 404 and insulation 406 can be used to suit a particular application. In one example, an embodiment of the drive shaft 404 has an outside diameter from about 0.10 inches to about 0.75 inches, and more particularly from about 0.15 inches to about 0.35 inches.

In the exemplary embodiment shown, the electrode 402 is a coiled wire. However, the electrode 402 may also be formed from coiled hypodermic tubing or may be a solid structure, such as a screw. The coil can have various shapes, such as conical, spherical, or any other shape suitable for ablation of a specific tissue. The electrode 402 may include a sharp distal tip 403 for penetrating a tumor tissue, as shown in FIG. 8. The size, shape, and materials used for the electrode 402 may vary to suit a particular application. For example, in one embodiment the electrode 402 may be made from stainless steel wire having a diameter from about 0.01 inches to about 0.1 inches. The electrode 402 may also be made from tungsten, titanium, or other suitable materials. The overall diameter (shown as “Q” in FIG. 7) of the electrode 402 may be from about 0.1 inches to about 1.5 inches. The length of the electrode 402 may include from about one to about ten coil turns, and may have an overall length (X4) of up to about 2.5 inches. In a particular embodiment, the overall length (X4) of the electrode coil 402 is about 0.30 inches. The electrode 402 may have a coil pitch (X5) of from about 0.05 inches to about 0.25 inches, with a left or right handed twist. It will be apparent that the dimensions may be varied depending on the application, the anatomy, or the size of the treated tissue. The overall length (X1) of the exemplary electrode assembly may be from about 4 inches to about 20 inches, and may vary to suit a particular application. In a more specific embodiment, the overall length (X1) is about 12.0 inches.

The electrode assembly 400 further may include a swiveling electrical connector 408 and a drive knob 410. The connector 408 is used to connect electrode assembly 400 with a power source or a control console. Generally, the control console may include a generator and indicators for monitoring performance of the thermal treatment device. The connector 408 may include a lock screw to prevent inadvertent loosening of the electrical connection. The drive knob 410 is used to rotate the electrode 402 clockwise or counter-clockwise to penetrate the tumor (FIG. 8). By turning the knob 410 a user can adjust the penetration depth of the electrode 402 within the tumor. The drive shaft 404 couples the electrode 402 to the knob 410 by transmitting to electrode 402 the rotational force applied to the knob 410. Knob 410 may have a knurled surface to improve gripping by the user or may include a rubber coating or similar structure to improve the user's grip.

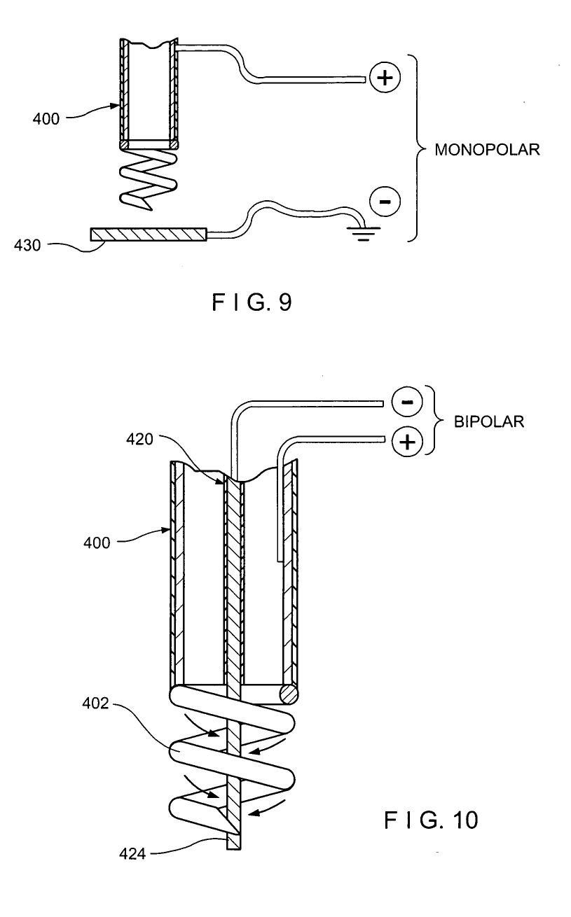

FIGS. 4 and 6 depict one exemplary embodiment of a negative electrode assembly 420 in accordance with the invention. The negative electrode assembly 420 is optional, as the thermal treatment device may be used in a monopolar mode without requiring a negative electrode, as depicted in FIG. 9. The electrode assembly 420 includes an electrode 424 covered by insulation 422. The materials used for the electrode 424 and insulation 422 can be any of those materials described with respect to the positive electrode assembly 400. The assembly 420 may further include an electrical connection 426 and a gripping portion 428. The electrical connection 426 may be used to connect the electrode assembly 420 with a power source or with a control console, which may be the same one to which the positive assembly 400 is connected. The electrical connection 426 may be soldered or may use other conventional connectors. The gripping portion 428 may be used for handling and positioning the assembly 420 by the user.

A distal tip portion of the negative electrode 420 having a length X3 is not insulated, for directing RF energy to the target tissue. The length X3 may be from about 0.06 inches to about 1.0 inches, and more particularly may be from about 0.10 inches to about 0.30 inches. The insulated portions 406, 422 of the two electrodes limit the thermal treatment range of the device's electrodes 402, 424. According to the invention, the distal tip 424 of the negative electrode 420 can be blunt or pointed, depending on the hardness of the tissue to be penetrated. The diameter of the negative electrode 424 may be from about 0.01 inches to about 1.0 inches, and more particularly from about 0.6 inches to about 0.9 inches. The overall length (X2) of the assembly 420 may be from about 6 inches to about 22 inches, and will vary to suit particular applications. For example, the overall length (X2) may be about 14.0 inches. In a particular embodiment, the negative assembly 420 may be longer than the positive assembly 400, so that the negative electrode 424 extends beyond the positive electrode 402. This configuration allows for directing the RF energy in a fashion that concentrates the treatment to the target tissue, while protecting the surrounding tissue from thermal damage.

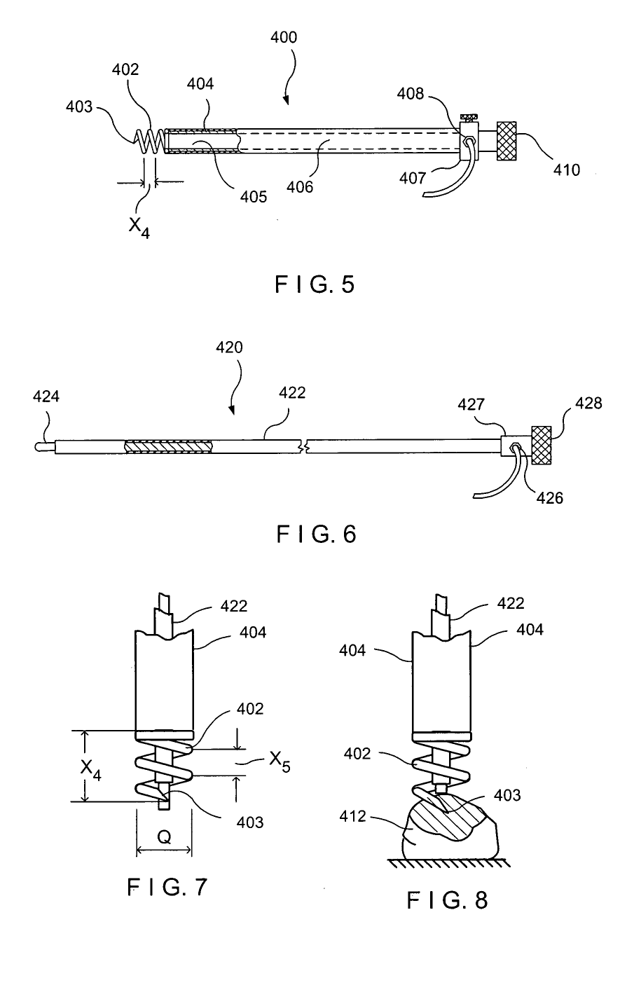

Referring to FIGS. 3, 4, 5, and 6, the assemblies 400, 420 may include hubs 407, 427 to facilitate interconnection between the positive and negative assemblies 400, 420. The hubs 407, 427 may also provide a sealing connection between the two components. In bipolar operation of the RF ablation device, shown in FIG. 10, the negative assembly 420 is positioned within the lumen 405 of positive assembly 400. Specifically, the negative electrode 424 is passed through the drive shaft 404 and extends through the positive coiled electrode 402. Alternatively, the relationship of the electrodes may be predetermined and the electrodes fixed in position with respect to each other. The hubs 407, 427 can be slidably positioned along the lengths of their respective assemblies 400, 420 to adjust the length of the negative assembly 420 that passes through the positive assembly 400. This also determines the length of the negative electrode 424 that extends beyond the positive electrode 402. In the exemplary embodiment, the assemblies 400, 420 are substantially rigid and are particularly well suited for open surgery or for laparoscopic procedures. Alternatively, the assemblies 400, 420 may be flexible laterally, as long as their coil and column strength are sufficient to allow for the transfer of torque and of longitudinal force necessary to pierce the tissue.

The mode of operation of an exemplary RF ablation electrode is shown with reference to FIG. 8. In the drawing, the sharp distal end 403 of the electrode 402 is shown beginning to penetrate a tumor 412. The tumor 412 is shown in partial cross-section to illustrate the distal end 403 of coil 402. In operation, the electrode 402 is disposed adjacent the tumor 412, and the electrode 402 is rotated so that the sharp distal end 403 penetrates the tissue of tumor 412. The electrode 402 is rotated by turning the knob 410 either clockwise or counter-clockwise, as necessary. As the user continues to turn the knob 410, the electrode 402 continues to penetrate the tumor 412 in a spiral fashion. Once the electrode 402 has been properly positioned, RF energy is applied to the tumor 412 until a desired level of ablation of the tumor has been achieved.

The operative components of an exemplary thermal ablation device are shown schematically in FIGS. 9 and 10. FIG. 9 depicts an exemplary embodiment using a monopolar mode of operation. In monopolar operation, only the positive assembly 400 is inserted in the tumor, and is used in conjunction with a grounding pad 430 or with another external grounding source. Alternatively, an independent internal grounding source, such as a secondary return electrode may be used in bipolar mode, as described below. In those embodiments where the positive electrode 402 comprises a solid structure, such as a screw, only monopolar operation is possible because the solid structure lacks a central lumen for receiving a return electrode. However, even a screw-like positive electrode may be fitted with a lumen sufficient for the passage of a negative electrode, thus enabling bipolar operation.

FIG. 10 depicts an exemplary RF ablation device using a bipolar mode of operation. Bipolar operation may be preferred when accurate targeting of the RF energy is important, since during monopolar operation the RF energy is not well targeted and may travel through tissues other than the target tissue. In this embodiment, the electrode 402 is inserted into the tumor, as previously described. In addition, by twisting the electrode 402 into the tumor the negative electrode 424 is also inserted into the tumor. In operation, current flows from the inside diameter of the coil electrode 402 and through the tumor tissue to the negative electrode 424. Alternatively, the polarity of the electrodes and thus the current flow can be reversed, such that the energy goes from the inner electrode to the outer coil electrode. Thermal treatment during bipolar operation is substantially confined to the area defined by the coil. Damage to surrounding healthy tissue is therefore much reduced compared to that resulting from monopolar operation.

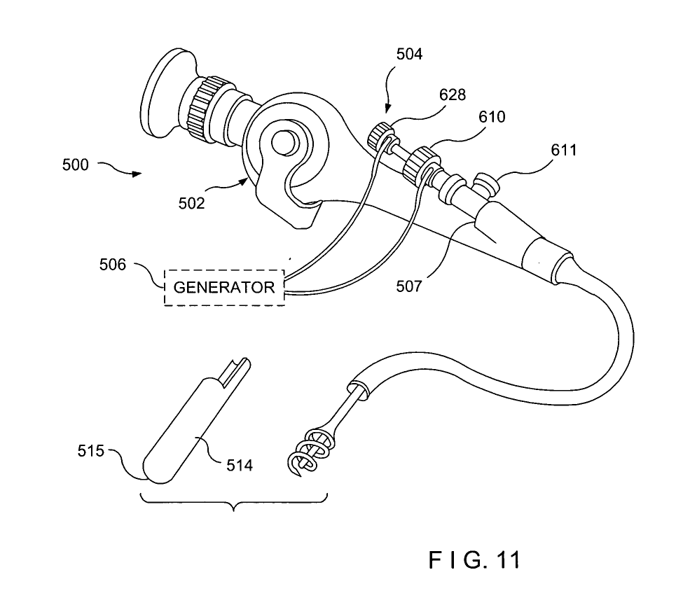

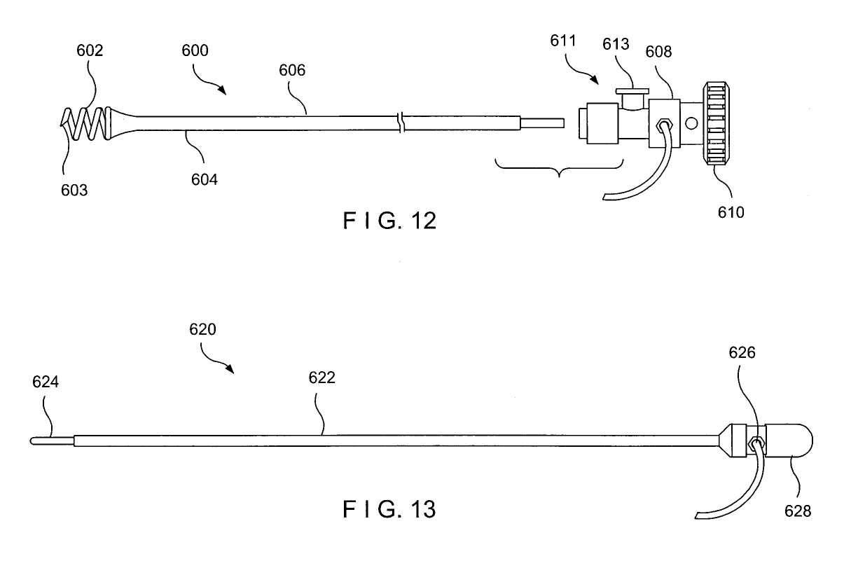

FIG. 11 depicts a scope assembly 500 in accordance with the invention, which includes a thermal treatment device with an electrode contained within the working channel of scope assembly 500. In the present embodiment, the scope 502 is a hysteroscope. However, the electrode assemblies described herein can be used with other types of scopes. The scope assembly 500 includes an electrode assembly 504, shown and described in detail with reference to FIGS. 12 and 13. The scope assembly 500 may also include an external power supply 506 used for powering the electrode assembly 504. FIGS. 12 and 13 show the positive electrode assembly 600 and the negative electrode assembly 620 prior to insertion in the scope 502. Use of the electrodes 600, 620 in conjunction with the scope 502 allows for direct visualization of the treatment site during the procedure, resulting in a potentially more effective and rapid treatment.

FIG. 12 depicts the positive electrode assembly 600 which may be used with a scope such as the hysteroscope 502 of FIG. 11. The assembly 600 has several similarities to the assembly 100 described above, and may include an electrode 602 having a sharp distal end 603, an insulated drive shaft 604, an electrical connector 608, and a drive knob 610. The electrode assembly 600 is configured to be used with a scope, for example by using a flexible drive shaft 604 and a connection 611 adapted to interface with a port of the scope. In the exemplary embodiment shown, the connection 611 is a luer lock connection that includes an extra port 613 used to facilitate the introduction of rinsing agents, drugs, or other therapeutic compounds to the thermal treatment site.

FIG. 13 depicts the negative electrode assembly 620 which may be use with a scope such as the hysteroscope 502 of FIG. 11. The negative electrode assembly 620 is similar to the negative electrode 420 described above, but is more specifically suited for use in conjunction with a hysteroscope. The negative electrode assembly 620 may include a partially insulated negative electrode 424, an electrical connection 626, and a gripping portion 628. Similarly to the positive assembly 600, the negative assembly 620 may include a flexible insulated shaft supporting the electrode at the distal end. The flexible shaft of the positive and negative electrodes 600, 620 allows the RF ablation assembly to follow the curves of the hysteroscope 502.

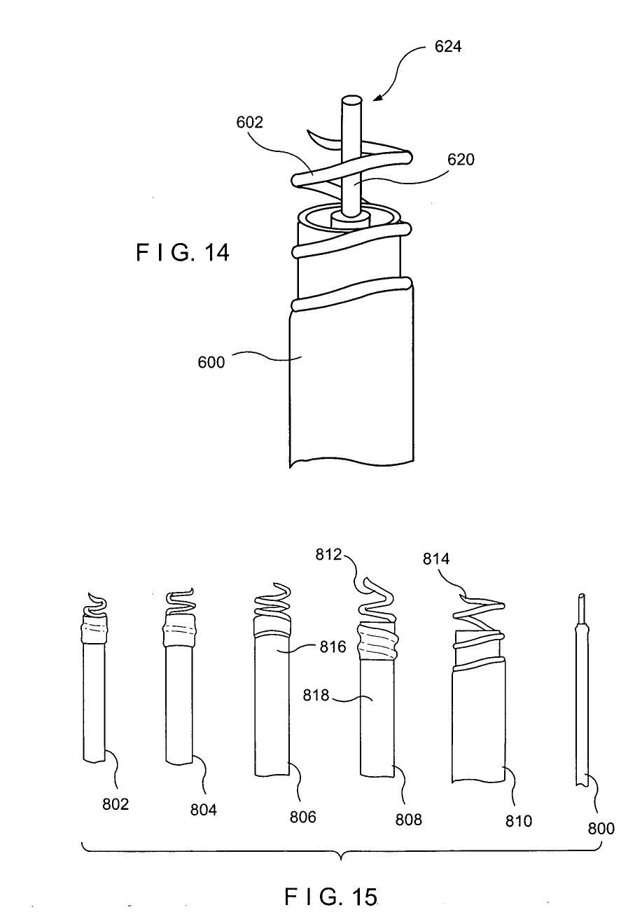

As shown in FIG. 11, the electrode assemblies 600, 620 are loaded into the scope 502 through a port 507 located in a proximal portion of the scope. The positive assembly 600 is coupled to the scope by the luer type fitting 611. The negative assembly 620 is inserted through the working channel of the positive assembly 600, and for example may be coupled thereto by using mating hubs. A drive knob 610 and gripping portion 628 protrude from the scope 502 and are accessible to the user to control and manipulate the device. In the embodiment shown, the assembly 500 includes an optional electrode protective structure 514. The structure can have a blunt end 515 that acts as a dilator. In one embodiment, the structure 514 is a sheath that covers the coil 602 during insertion, for example to dilate surrounding tissue, and can break away to expose the electrode 602 for insertion into the tumor. FIG. 14 shows a pictorial representation of the tip 624 of negative electrode 620 and of coil 602 of positive electrode 600 as they appear without the protective structure 514.

In general, it may be desirable to use large electrodes to carry out RF ablation because the electrode size is related to the size of the defect created in the tissue by the thermal treatment. A larger electrode will produce a larger area of affected tissue. However, the use of large coil electrodes in laparoscopic procedures may be limited by the size of the trocar access port utilized, through which the electrodes must pass. Hysteroscopic access may also be limited to electrodes that fit through a working channel of a rigid or flexible hysteroscope or other type of scope. Therefore, in order to utilize larger diameter electrodes and avoid the aforementioned drawbacks, the electrode may be front loaded through the working channel of the scope's distal end before inserting the assembly into a patient. In this exemplary embodiment according to the present invention, the positive electrode may be larger than the working channel and other passages of the insertion apparatus, since it does not have to travel therethrough.

FIG. 18 shows an exemplary embodiment of a front loaded positive electrode. As discussed above, positive electrode 700 has a larger diameter than would be possible if the electrode were required to pass through the working lumen of a catheter. Positive electrode 700 comprises an insulated shaft portion 702 which extends partially into a catheter's distal end to secure the electrode in place. Insulated shaft portion 702 may be made of a conductive material or may include separate conductors to provide power to the conductive coil 706. Coil 706 may be similar to the positive conductor coils described above, and may have a shape and size appropriate for the procedure being performed. A torque transferring connector 708 may be used to attach coil electrode 706 to the insulated shaft 702. The proximal end 704 of insulated shaft 702 may comprise an electrical connection which interfaces with a corresponding connection in the catheter. A mechanical connection may also be present at proximal end 704, to transmit torque to the shaft 702, and assure that the positive electrode 700 is not prematurely released from the introducing catheter (such as the connection 611 shown in FIG. 12 that is connectable to the electrode at the proximal end after it is front loaded into the scope).

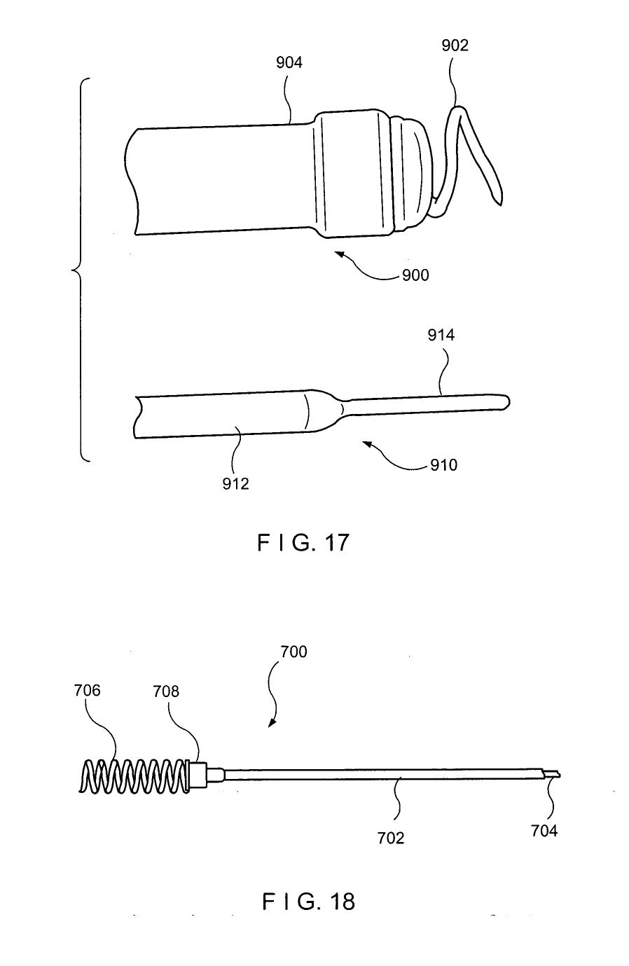

It will be apparent to those of skill in the art that the positive and negative electrodes of the RF thermal ablation device according to the invention may take different shapes. For example, FIG. 15 shows five different positive electrodes and one negative electrode which may be used to ablate different types of tissue. Positive electrodes 808 and 810 have a larger diameter than positive electrodes 802-806, and thus would be recommended to treat larger masses of tissue. The pitch of the distal coil of the electrodes may be varied, to penetrate tissue masses having different densities. For example, electrode 808 has a greater pitch between loops of coil 812. The thickness of the coils and the sharpness of the coil's distal tip (for example tip 814) may be varied to optimize the device to penetrate different tissues. Any of the positive electrodes shown may be used in conjunction with negative electrode 800. A working channel or lumen is provided within the shaft of the positive electrode (for example shafts 816, 818) to form a passage for the negative electrode 800, or for a similar element. FIG. 17 shows an additional embodiment of the RF ablation device including a positive electrode 900 and a negative electrode 910. Positive electrode 900 comprises the insulated shaft 904 and the conductive coil 902. Negative electrode 910 comprises the insulated shaft 912 and a conductive tip 914 adapted to extend from the center of coil 902.

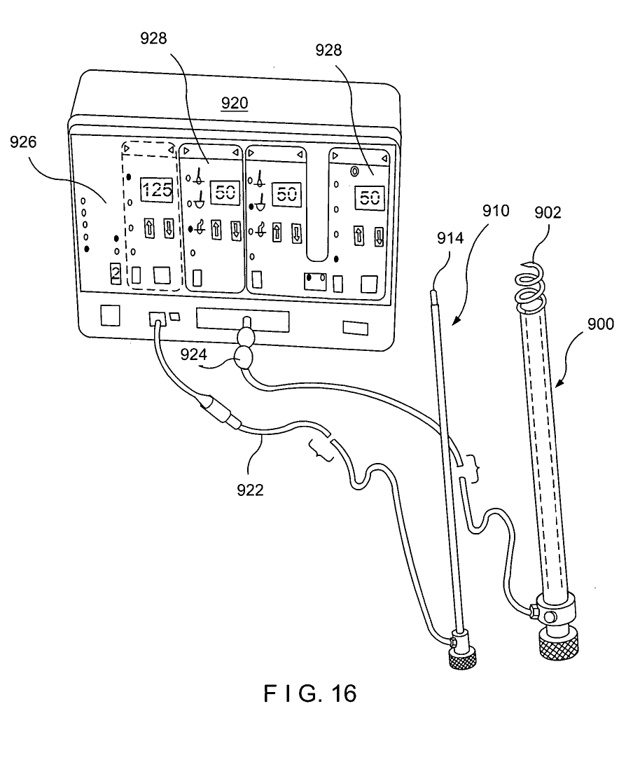

FIG. 16 depicts an embodiment of the RF thermal ablation device of the present invention in a configuration ready to be used. The exemplary positive electrode 900 and negative electrode 910 are connected to a control console 920 which is adapted to provide power to the device. A positive connector 924 may be used to connect positive electrode 900, while a negative connector 922 may connect negative electrode 910. Both power and monitoring signals may be carried by the connectors 922, 924, so that control console 920 may be used also to monitor the performance of the device. A control panel 926 may be provided, for example to select the voltage and/or current flowing to the electrodes. One or more monitoring panels 928 may also be provided, to ascertain the effectiveness of the treatment provided by the exemplary ablation device. For example, the current flowing through the affected tissue may be monitored, to note any change in the tissue's impedance. Alternatively, or concurrently, one or more temperature monitors may be used with the electrodes to monitor the temperature of th target tissue as it is treated.

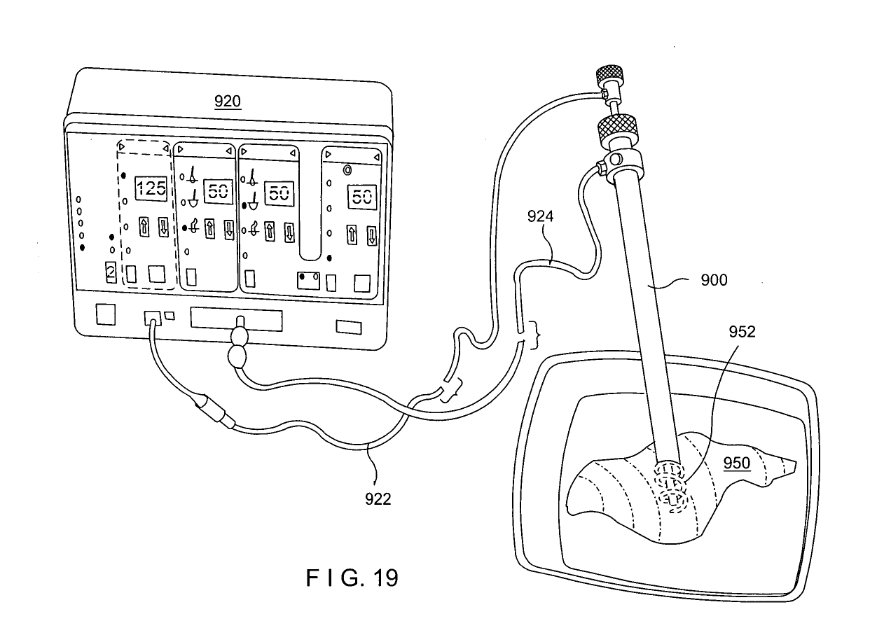

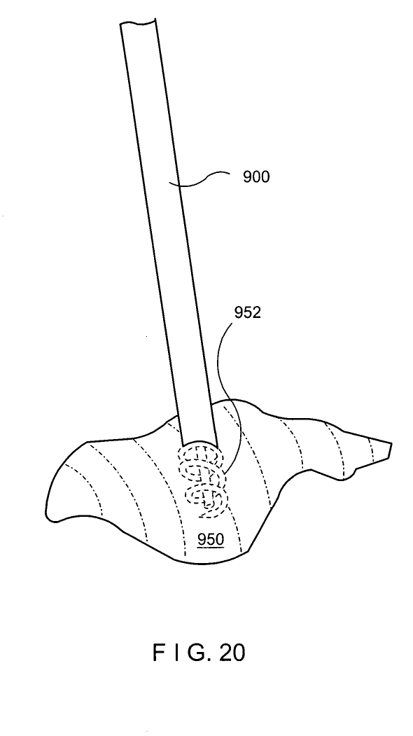

An exemplary application of the thermal ablation device according to the invention is depicted in FIGS. 19 and 20. In the example, a target tissue 950 (in this case chicken tissue) was ablated using the ablation device formed by the positive electrode 900 and negative electrode 910. Negative electrode 910 was inserted into the working channel of a positive electrode 900 and is not visible. A control console 920 was used to select the voltage, current and other parameters to optimize the ablation process. After a certain amount of time during which the ablation was carried out, a region of ablated tissue 952 became visible. The duration of the ablation process may be controlled by visually monitoring the size of the region of ablated tissue 952 using, in this case, the optics of a scope through which the ablation catheters 900, 910 were inserted. Alternatively, measurements of the region of tissue may be made to determine changes in the tissue's properties. For example, conductivity, light transmission or other tissue properties may be monitored, to determine when the desired level of ablation has been achieved.

The present invention has been described with reference to specific exemplary embodiments. Those skilled in the art will understand that changes may be made in details, particularly in matters of shape, size, material and arrangement of parts. Accordingly, various modifications and changes may be made to the embodiments. Additional or fewer components may be used, depending on the condition that is being treated using the described self anchoring RF ablation device. The specifications and drawings are, therefore, to be regarded in an illustrative rather than a restrictive sense.

What is claimed is:

1. A tissue treatment device comprising:
a handle;
a tissue anchoring portion operatively connected to the handle, the tissue anchoring portion forming an electrode of a first polarity; and
a plurality of arms deployable in proximity to the anchoring portion, at least one of the arms comprising a second electrode of a second polarity.

2. The tissue treatment device according to claim 1, wherein the tissue anchoring portion comprises a coil for penetrating and stabilizing tissue.

3. The tissue treatment device according to claim 2, further comprising a shaft extending from the handle to the tissue anchoring portion, wherein the coil is deployable from a distal end of the shaft and retractable thereinto.

4. The tissue treatment device according to claim 1, further comprising a shaft extending from the handle to the tissue anchoring portion, wherein the plurality of arms is deployable from a distal end of the shaft and retractable thereinto.

5. The tissue treatment device according to claim 4, wherein the tissue anchoring portion comprises a coil for penetrating and stabilizing tissue and wherein the plurality of arms is deployable along a longitudinal axis of the coil.

6. The tissue treatment device according to claim 2, wherein the at least one of the arms comprising the second electrode which cooperates with the coil to define a shape of an effective region of the treatment device.

7. The tissue treatment device according to claim 4, wherein the plurality of arms is deployable from a hollow core of the shaft.

8. The tissue treatment device according to claim 1, wherein the plurality of arms comprises an array of tines.

9. A system for ablating target tissue in a body, comprising:
an elongated shaft having a distal end insertable into the body to abut the target tissue;
a coil extending from the distal end, anchorable to the target tissue and forming a first electrode of a first polarity;
an array of a plurality of tines at the distal end, at least one of the tines forming a second electrode of a second polarity; and
an electric power supply connectable to the coil and to the array of tines for creating an electric potential difference therebetween.

10. The system according to claim 9, wherein the array of tines is deployed and extends through the coil along a longitudinal axis thereof.

11. The system according to claim 9, wherein the array of tines extends beyond a distal end of the coil, curving around the distal end of the coil.

12. The system according to claim 9, wherein each of the tines forms a second electrode and wherein the coil and the array of tines cooperate to form a bipolar system with an effective region of tissue treatment defined by the relative positions of the tines and the coil.

13. The system according to claim 9, further comprising a handle coupled to a proximal end of the shaft.

14. The system according to claim 10, wherein the shaft comprises a control portion at a distal end thereof from which the array of tines is deployed.

15. The system according to claim 9, further comprising a switch to electrically connect the power supply to the coil and to the array of tines.

16. The system according to claim 9, wherein the array of tines is movable between a first position withdrawn into a hollow core of the elongated shaft and a second position extended therefrom.

17. The system according to claim 16, wherein, in the second position, the array of tines assumes a substantially umbrella-shaped configuration.

18. A method of ablating target tissue, comprising:
positioning a distal end of an elongated shaft so that it abuts the target tissue;
anchoring the distal end in the target tissue by actuating an anchoring portion of the elongated shaft, the anchoring portion comprising a first electrode;
deploying a second electrode from the distal end of the shaft to contact the target tissue; and
applying an electric potential between the first electrode of the anchoring portion and the second electrode.

19. The method according to claim 18, further comprising the step of, after the tissue has been ablated, withdrawing the negative electrode and detaching the anchoring portion from the target tissue to remove the distal end from the body lumen.

20. The method according to claim 18, wherein deploying the second electrode comprises deploying an array of tines.

21. The method according to claim 18, further comprising screwing a coil of the anchoring portion into the target tissue to anchor the distal end of the elongated shaft relative to the target tissue.

22. The method according to claim 20, further comprising deploying the array of tines in an umbrella-like configuration surrounding a distal end of the anchoring portion.

23. The method according to claim 18 further comprising the step of inserting the distal end of the elongated shaft into a body lumen via a naturally occurring body orifice to reach the target tissue.

24. The method according to claim 20, further comprising the step of configuring a deployed position of the array of tines to cooperate with a coil of the anchoring portion in defining a desired effective region of tissue treatment.

25. The method according to claim 18, wherein the distal end of the elongated shaft is inserted through a trocar to abut the target tissue.

26. The method according to claim 25, wherein the elongated shaft is inserted through the trocar under laproscopic guidance.

27. The method according to claim 18, wherein the distal end of the elongated shaft is inserted percutaneously under laproscopic guidance to abut the target tissue.

28. A thermal treatment apparatus comprising:
a positive electrode assembly adapted for insertion in a body lumen;
an elongated shaft of the positive electrode assembly;
a coil-like electrode mounted distally on the positive electrode assembly;
a negative electrode assembly having an elongated shaft inserted in a working channel of the positive electrode assembly; and
an electrode mounted distally on the negative electrode assembly, the electrode extending through the coil-like electrode.

29. The thermal treatment apparatus according to claim 28, further comprising a control console connected to the positive and negative electrode assemblies.

30. The thermal treatment apparatus according to claim 29, further comprising a power source of the control console.

31. The thermal treatment apparatus according to claim 29, further comprising monitoring and power control instruments of the control console.

32. The thermal treatment apparatus according to claim 28, wherein the elongated shaft of the negative electrode assembly is adapted to slide longitudinally in the working channel of the positive electrode assembly.

33. The thermal treatment apparatus according to claim 28, further comprising a hub adapted to connect the positive electrode assembly to the negative electrode assembly.

34. The thermal treatment apparatus according to claim 28, further comprising a proximal grasping portion of the positive electrode assembly.

35. The thermal treatment apparatus according to claim 28, further comprising a proximal grasping portion of the negative electrode assembly.

36. The thermal treatment apparatus according to claim 28, wherein the electrode mounted distally on the negative electrode assembly comprises a longitudinal non insulated elongated protrusion.

37. The thermal treatment apparatus according to claim 34, wherein the coil-like electrode has an inner diameter greater than a diameter of the elongated longitudinal protrusion.

38. The thermal treatment apparatus according to claim 28, wherein the positive electrode assembly comprises a handle to transmit torque to the coil-like electrode.

39. The thermal treatment apparatus according to claim 28, wherein the elongated shaft of the positive electrode assembly is adapted for front loading in a medical insertion device.

40. The thermal treatment apparatus according to claim 39, wherein the medical insertion device is an hysteroscope.

41. The thermal treatment apparatus according to claim 28, wherein the coil-like electrode is one of a compression spring, a screw and a corkscrew shaped coil.

42. The thermal treatment apparatus according to claim 28, wherein the coil-like electrode comprises a distal sharp point.

43. The thermal treatment apparatus according to claim 36, wherein the elongated protrusion comprises a sharp distal end.
